(19) United States
(12) Patent Application Publication
(10) Pub. No.: US 2021/0079332 A1
(43) Pub. Date: Mar. 18, 2021

(54) CELL CULTURE CARRIER

(71) Applicant: TECHNISCHE UNIVERSITAET ILMENAU, Imenau (DE)

(72) Inventors: Andreas SCHOBER, Erfurt (DE); Frank WEISE, Ilmenau (DE); Joerg HAMPL, Erfurt (DE)

(21) Appl. No.: 16/954,035

(22) PCT Filed: Dec. 15, 2017
(86) PCT No.: PCT/EP2017/083151

§ 371 Date: Jun. 15, 2020

(51) Int. Cl. C12M 1/12

(52) U.S. Cl. CPC C12M 25/04

(57) ABSTRACT

The invention relates to a cell culture carrier for cultivating biological cell material (07). The cell culture carrier comprises: a carrier plate (01); a membrane (02), which is supported by the carrier plate (01) and provides a colonization surface (06) for the cell material (07), the colonization surface (06) being permeable for a main flow (10) of a nutrient solution; and a holding cage (03), which is covered by the membrane (02) at an end and into which the cell material (07) can be introduced and through which the main flow (10) can flow. The carrier plate (01) provides a plurality of flow openings (08), which are positioned outside of the holding cage (03) in peripheral distribution and which allow a secondary flow (11) of the nutrient solution, the flow velocity of which secondary flow is greater than the flow velocity of the main flow (10) of the nutrient solution that flows through the holding cage (03) and the colonization surface (06). The invention further relates to a cell culture carrier assembly having a cell culture carrier of this type in a housing, which provides a flow region for a nutrient solution.

### BACKGROUND OF THE INVENTION

The invention relates to a cell culture carrier for cultivating biological cell material. The cell culture carrier has a carrier plate, which is preferably adapted to external units, in which a large number of such cell culture carriers are usually combined and supplied with the media required for cell cultivation. The cell culture carrier also has a porous membrane which is carried by the carrier plate and is preferably designed as a structured molded body. The membrane is permeable to a main flow of a nutrient solution and provides a colonization surface for the cell material to be cultivated. Furthermore, the cell culture carrier comprises a receiving cage or a comparable cavity into which the cell material can be introduced. The receiving cage is arranged on the membrane, inserted therein or formed in one piece therewith, such that an end face of the receiving cage is covered by the membrane in order to hold the cell material in the cage. The main flow flows through the receiving cage during operation in order to supply the cell material with nutrients, oxygen or the like.

Biological cell material, e.g. pieces of tissue, biopsy material or multicellular tissue-like cell clusters, must be cultivated in life-promoting conditions if they are to be examined and, if necessary, multiplied outside of their natural environment over a medium-term period of several hours to a few weeks. For this purpose, they are placed on cell culture carriers and supplied with the appropriate media or nutrients, with a fluid perfusing the cell material in order to introduce nutrients and dissolved oxygen to the individual cells. In the simplest case, the cell materials are deposited in the wells of microtiter plates, which can be equipped with special inserts.

DE 20 2006 017 853 U1 describes an insert for a microtiter plate, consisting of a carrier structure in which at least one depression is made, the upper diameter of the depression being selected such that it can be inserted into a depression in the microtiter plate. The bottom of each depression has at least one microcavity that is shaped downwards. The insert is provided with pores at least in part.

In addition to microtiter plates, there are other approaches for providing cavities for the cultivation and examination of biological substances.

For example, WO 2011/035937 A1 describes a microstructured molded body comprising a film which is divided into undeformed regions and thinned stretching regions. Microstructures are formed at least in some of the thinned stretching regions, pores being formed in at least one of the thinned stretching regions and at least some of the undeformed regions being impermeable.

Furthermore, WO 2011/035938 A1 discloses a microstructured molded body which has a film-like main body which comprises a first film layer and a second film layer located underneath, the second film layer having recesses having a diameter of less than 2 mm, which are formed by deformed regions of the first film layer, by means of which cavities are formed. At least some of the deformed regions of the first film layer have pores. The regions of the film-like base body are impermeable outside the recesses.

DE 10 2010 037 968 A1 describes a structure for simulating a sinusoid, which can be inserted into a microtiter plate. The structure comprises a plurality of layers of a porous material arranged one above the other, a space being formed between each of the layers. The spaces are connected by channels formed in the layers for conveying a fluid.

However, it has been found that a high transport rate of the nutrients (including dissolved gases, in particular oxygen) is required for supplying certain cell materials. The easiest way to achieve this is by means of high flow rates of the medium containing the nutrients. However, a high flow rate results in high shear forces in the cell clusters, and the sensitive tissue regions of said clusters cannot withstand these forces. This ultimately results in the cell material being destroyed. In the prior art, attempts are known to prevent the shear-force-related dissolution of a cell assembly by enclosing it in a receiving cage, but this does not solve the problem described. Either a tightly closed cage prevents the desired high transport rate or a wide open cage cannot adequately protect the cell material.

Proceeding from this problem, the problem addressed by the invention is to provide an improved cell culture carrier by means of which the biological cell material thereon can be cultivated in the medium term, with the transport rate of the required nutrients being intended to be increased significantly without increased flow rates of the nutrient solution resulting in dissolution or destruction of the cultivated cell clusters.

### SUMMARY OF THE INVENTION

This problem is solved by a cell culture carrier according to the appended claim 1 and by a cell culture carrier assembly according to claim 10.

The cell culture carrier according to the invention is characterized in that the carrier plate provides a plurality of flow openings which are distributed over the circumference outside the receiving cage. The plurality of flow openings allow for a secondary flow of the nutrient solution, the flow rate of which is greater than the flow rate of the main flow of the nutrient solution flowing through the receiving cage and the colonization surface.

The receiving cage in turn provides a porous container for the cell material. The wall of the receiving cage preferably consists entirely of porous material, which allows the nutrient solution to flow through. The end faces of the receiving cage are preferably completely open, the lower end face being closed by the membrane in order to hold the cell material in the receiving cage. The upper end face, on the other hand, can remain open to enable the cell material to be inserted and, if necessary, to also allow for visual observations. The main flow of the nutrient solution flows axially through the receiving cage and ensures the basic supply of the cells. In addition, the secondary flow is guided along the outside of the receiving cage, such that nutrient transport can also take place via the porous side walls of the receiving cage. Due to the higher flow rate of the secondary flow compared with the main flow, the transport rate for the nutrients is high, because there is a high gradient of nutrients, oxygen and cell degradation products between the inside of the receiving cage and the outside supplied by the secondary flow, which, despite the barrier effect of the cage, allows cultivated cells to be supplied in an improved manner. The cell material remains protected within the receiving cage and there is a lower flow rate.

According to an advantageous embodiment, the receiving cage is made of the same porous material as the membrane, for example of polycarbonate. The pore size in the wall of the receiving cage can also be selected to be identical to the membrane, which results in favorable dynamic pressure conditions within the receiving cage.

The receiving cage is preferably formed as a sleeve having open end faces, in particular having a cylindrical shape. However, rectangular or polygonal cross sections can also be selected for the receiving cage.

A preferred embodiment is characterized in that the flow openings are designed as gaps between the membrane and the carrier plate. The flow openings can thus be placed particularly close to the outside of the wall of the receiving cage, such that the secondary flow can be guided along the outside at a high and uniform flow rate. Alternatively, the flow openings can also be made in the form of a plurality of circular portions in the carrier plate or can be formed on the outer circumference thereof by corresponding cut-outs.

According to a developed configuration, the carrier plate has one or more holding means for fastening the cell culture carrier within a bioreactor. For example, locking lugs or the like can be used to anchor the cell culture carrier in a well of a microtiter plate. For this purpose, the carrier plate is shaped, for example, as a circular disk, the dimensions of which are adapted to the wells provided in microtiter plates. The cell culture carrier can also be used in tubular housings or tubular sleeves.

It is advantageous for the membrane to be enclosed in its edge region on all sides by the carrier plate and to be fastened to the carrier plate by a plurality of holding webs which are distributed over the circumference. The membrane can thus be held and protected in a simple manner, and at the same time the formation of the main and secondary flow is ensured.

According to a preferred embodiment, the complete cell culture carrier consists of polycarbonate. Other biocompatible materials can also be used.

The invention also relates to a cell culture carrier assembly for cultivating biological cell material. In addition to said cell culture carrier, the cell culture carrier assembly comprises a housing which provides a flow region for a nutrient solution. The carrier plate, the membrane and the receiving cage are positioned in the flow region of the housing. There are a plurality of flow openings within the housing which are distributed over the circumference outside the receiving cage and allow for the secondary flow of the nutrient solution, the flow rate of which is greater than the flow rate of the main flow of the nutrient solution flowing through the receiving cage and the colonization surface.

### BRIEF DESCRIPTION OF THE DRAWINGS

Further advantages and details of the invention result from the following description of a preferred embodiment with reference to the drawings, in which:

FIG. 1 is a plan view of a cell culture carrier according to the invention;

FIG. 2 is a side view of the cell culture carrier according to FIG. 1;

FIG. 3 is a perspective sectional view of the cell culture carrier according to FIGS. 1 and 2.

### DETAILED DESCRIPTION OF THE INVENTION

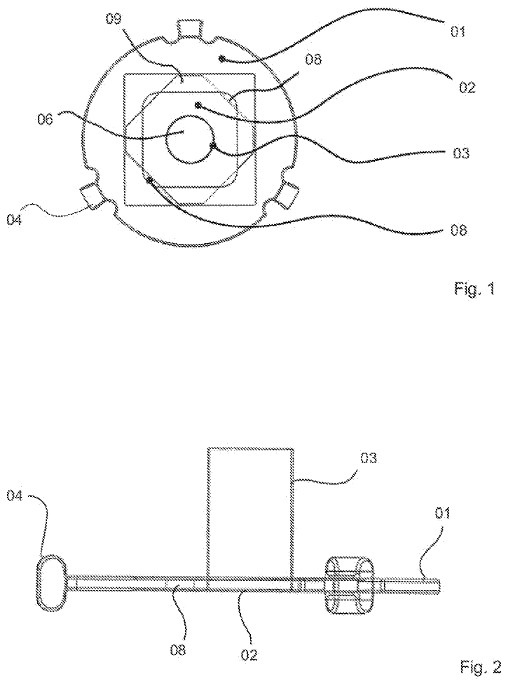

An exemplary embodiment of a cell culture carrier according to the invention is shown in FIGS. 1 to 3. The cell culture carrier has a carrier plate 01, a porous membrane 02 and a receiving cage 03.

The carrier plate 01 is designed here as a circular disk made of polycarbonate having a diameter of approx. 15 mm and has, on its outer edge, holding means 04 by means of which the entire cell culture carrier can be fastened in a housing (not shown). The membrane 02 is also produced as a structure made of polycarbonate and is clamped at its edges in the carrier plate 01. A colonization surface 06 is provided in the center of the membrane 02, on which cell material 07 can be deposited. The membrane is porous at least in the region of the colonization surface 06, such that a nutrient solution can flow through the pores.

The cylindrical receiving cage 03 stands up on the membrane 02 in the region of the colonization surface 06 and is fastened to said membrane. In other configurations, the receiving cage can be inserted into the membrane or can be formed in one piece therewith. The receiving cage 03 can also be made of polycarbonate and has, for example, a diameter of 3 mm and a height of 5 mm. The side walls of the receiving cage 03 have a thickness of approx. 50 μm and are also permeable, preferably porous, such that nutrient solution or its components can also penetrate there. The upper end face of the receiving cage 03 is open in order for it to be possible to insert and remove the cell material 07 at this point.

Clearances are provided in the corner regions of the membrane 02, such that a plurality of flow openings 08 remain in the carrier plate 01. The flow openings 08 are distributed over the circumference outside the receiving cage 03. Holding webs 09 for holding the membrane remain between the membrane 02 and the carrier plate 01. In different configurations, the flow openings can be formed by a multi-part circumferential annular gap.

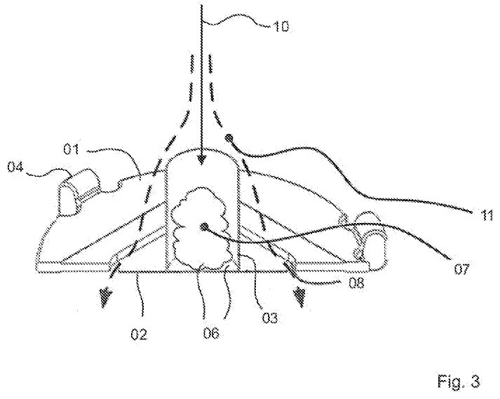

FIG. 3 shows the flow profile generated by the described construction for the nutrient solution by means of flow arrows. A main flow 10 flows directly through the interior of the receiving cage 03, perfuses the cell material 07 in the process, and emerges from the membrane 02 again. The pore size in the colonization surface 06 and the pressure of the main flow 10 are selected such that the flow rate is not too high, and therefore the cell material 07 is not damaged by shear forces that occur. A secondary flow 11 passes the outside of the receiving cage 03 and flows out through the flow openings 08. Since the secondary flow does not act directly on the cell material 07, its flow rate can be selected to be significantly higher. Owing to the higher flow rate, a high gradient of the nutrients or the gases dissolved in the nutrient solution occurs between the inside of the receiving cage 03 and its outside, such that they are transported through the porous wall of the receiving cage 03. The flow rate of the secondary flow 11 can be determined, for example, by appropriately selecting the cross section provided by the flow openings 08. The cross section of the flow openings 08 is generally larger in total than the sum of the cross section of the pores in the region of the colonization surface 06.

1. Cell culture carrier for cultivating biological cell material, comprising:
a carrier plate;
a membrane which is carried by the carrier plate and provides a colonization surface for the cell material, the colonization surface being permeable to a main flow of a nutrient solution;
a receiving cage which is permeable to the nutrient solution and is covered on an end face by the membrane, into which the cell material can be introduced and through which the main flow can flow;
characterized in that the carrier plate provides a plurality of flow openings which are distributed over the circumference outside the receiving cage and allow for a secondary flow of the nutrient solution, the flow rate of which is greater than the flow rate of the main flow of the nutrient solution flowing through the receiving cage and the colonization surface.

2. Cell culture carrier according to claim 1, the receiving cage consists of the same porous material as the membrane.

3. Cell culture carrier according to claim 2, wherein the receiving cage is shaped as a sleeve having open end faces, with one end face standing up on the membrane and the opposite end face remaining open for introducing the cell material and for feeding in the main flow of the nutrient solution.

4. Cell culture carrier according to claim 1, wherein the flow openings are designed as gaps between the membrane and the carrier plate.

5. Cell culture carrier according to claim 1, wherein the carrier plate has holding means for fastening within a bioreactor.

6. Cell culture carrier according to claim 1, wherein the membrane is enclosed in its edge region by the carrier plate and is fastened to the carrier plate by a plurality of holding webs which are distributed over the circumference.

7. Cell culture carrier according to claim 1, wherein the carrier plate is shaped as a circular disk, the dimensions of which are adapted to the wells provided in microtiter plates such that the cell culture carrier can be inserted into such a well.

8. Cell culture carrier according to claim 7, wherein the membrane and the wall of the receiving cage have a thickness of between 25 and 75 μm, in that the receiving cage has an axial length of between 2.5 and 3.5 mm, in that the circular carrier plate (01) has a diameter of between 4 and 18 mm, and in that the flow openings together have a cross section of between 0.5 and 10 mm².

9. Cell culture carrier according to claim 1, wherein it consists entirely of polycarbonate.

10. Cell culture carrier assembly for cultivating biological cell material, comprising:
a housing which provides a flow region for a nutrient solution;
a carrier plate which is positioned in the flow region of the housing;
a membrane which is carried by the carrier plate and provides a colonization surface for the cell material, the colonization surface being permeable to a main flow of the nutrient solution;
a receiving cage which is permeable to the nutrient solution, is arranged on the membrane and into which the cell material can be introduced;
characterized in that there are a plurality of flow openings within the housing which are distributed over the circumference outside the receiving cage and allow for a secondary flow of the nutrient solution, the flow rate of which is greater than the flow rate of the main flow of the nutrient solution flowing through the receiving cage and the colonization surface.
